(19) United States
(12) Patent Application Publication
(10) Pub. No.: US 2026/0076583 A1
(43) Pub. Date: Mar. 19, 2026

(54) BIOIMPEDANCE MEASUREMENT CIRCUIT, METHOD AND ELECTRONIC DEVICE

(71) Applicant: ams Sensors Germany GmbH, Jena (DE)

(72) Inventors: Julio SALDANA, Jena (DE); Parvathy SASIKALA JAYACHANDRAN PILLAI, Erfurt (DE); Predrag MICAKOVIC, Erfurt (DE)

(21) Appl. No.: 19/106,554

(22) PCT Filed: Aug. 23, 2023
(86) PCT No.: PCT/EP2023/073091

§ 371 Date: Feb. 25, 2025

(30) Foreign Application Priority Data
Aug. 30, 2022 (DE) 10 2022 121 888.0
Nov. 22, 2022 (DE) 10 2022 130 841.3

(51) Int. Cl. A61B 5/0531; A61B 5/25

(52) U.S. Cl. CPC A61B 5/0531; A61B 5/25; A61B 2560/0223

(57) ABSTRACT

A bioimpedance measurement circuit for determining a corrected body impedance includes terminals, a control circuit, an evaluation circuit and a signal processing circuit. The control circuit controls measurement of a body impedance, where stimulus current is applied through a first and second terminal, and an input voltage is measured between a third and fourth terminal, of a first bodypart impedance, where the stimulus current is applied through the first and third terminal, and the input voltage is measured between the first and third terminal, and of a second bodypart impedance, where the stimulus current is applied through the second and fourth terminal, and the input voltage is measured between the second and fourth terminal. The signal processing circuit determines an error impedance based on the measured bodypart impedances and stored parasitic/input impedances and determines a corrected body impedance based on the measured body impedance and the error impedance.

### RELATED APPLICATIONS

This patent application is a national stage application filed under 35 U.S.C. 371 based on International Patent Application No. PCT/EP2023/073091, filed Aug. 23, 2023, which claims the priority of German patent applications 102022121888.0 filed Aug. 30, 2022 and 102022130841.3 filed Nov. 11, 2022, wherein the disclosures of all prior applications are hereby incorporated by reference in their entireties.

### TECHNICAL FIELD

The disclosure relates to a bioimpedance measurement circuit for measuring a bioimpedance across measurement electrodes, and to an electronic device with such bioimpedance measurement circuit. The disclosure further relates to a corresponding bioimpedance measurement method and to a computer program product therof.

### BACKGROUND

Bioimpedance measurement is a biomedical technique to determine the electrical behavior of living tissue by using an electrode arrangement for applying a stimulus current to a sample, for example a part of a human body, and measure the resulting voltage through a four point measurement method.

The amplitude and phase of the resulting voltage signal will depend on the BIOZ impedance. The bioimpedance of interest may be determined by the measured voltage divided by the stimulus current injected into the tissue.

Due to asymmetries in a current path of the arrangement of the measuring electrodes to perform the four point measurement method, a common mode signal is created across the body impedance. Those asymmetries arise because of a mismatch between the skin/electrode contact impedances. Moreover, due to asymmetries in the voltage path between the measuring electrodes and a differential amplifier to evaluate the voltage across the measuring electrodes, the common mode signal is translated to an extra differential signal that is added to the desired differential signal. As a result, the measured magnitude of the total input differential signal which is applied to the differential amplifier is higher than the expected one and the phase is also affected.

### SUMMARY

The present disclosure provides an improved bioimpedance measurement concept which provides the bioimpedance of interest as accurately as possible, and in particular attenuates effects of mismatch between the skin/electrode contact impedances.

Bioimpedance measurement usually is performed by applying a current to the sample and measure the generated voltage through a 4 point measurement method. The applied current is an alternating current, for example a 50 Khz sine wave.

The amplitude and phase of the resulting voltage signal will depend on the bioimpedance or, in general, body impedance.

The improved bioimpedance measurement concept is based on the finding that the actual measurement of the body impedance is affected by errors due to unavoidable mismatch in contact impedances resulting in common mode signals. Hence a correction is proposed that is based on various contact impedances being effective during application of the four electrodes onto the body. However, since even these impedances are subject to parasitic impedances, a correction of the measured body impedance to determine a corrected body impedance needs to be based on various impedances of body parts and corresponding stored parasitic impedances, which may be determined during a calibration phase. Hence for each measurement of a body impedance at least two bodypart impedance measurements have to be performed, the bodyparts e.g. corresponding to a location, where the electrodes are applied, like a wrist and a finger, in case of e.g. usage in a wristband or watch on a wrist.

In an embodiment of a bioimpedance measurement circuit according to the improved bioimpedance measurement concept the bioimpedance measurement circuit comprises a set of terminals comprising a first terminal for connecting a first electrode, a second terminal for connecting a second electrode, a third terminal for connecting a third electrode and a fourth terminal for connecting a fourth electrode, each electrode to be attached to a body. A control circuit is included to control application of a stimulus current with a measurement frequency, in particular an AC current, through a first subset of two selected terminals of the set of terminals and measurement of an input voltage in response to the stimulus current at a second subset of two selected terminals of the set of terminals. An evaluation circuit is included for determining a measured impedance in response to the stimulus current and the measured input voltage. A signal processing circuit determines a body impedance.

The control circuit is configured to control measurement of a body impedance, where the stimulus current is applied through the first and the second terminal, and the input voltage is measured between the third and the fourth terminal, of a first bodypart impedance, where the stimulus current is applied through the first and the third terminal, and the input voltage is measured between the first and the third terminal, and of a second bodypart impedance, where the stimulus current is applied through the second and the fourth terminal, and the input voltage is measured between the second and the fourth terminal. The signal processing circuit is configured to determine an error impedance based on the measured first bodypart impedance, the measured second bodypart impedance, a stored first parasitic bodypart impedance, a stored second parasitic bodypart impedance, and a stored input impedance, and to determine a corrected body impedance based on the measured body impedance and the error impedance.

Hence with only two additional measurements, i.e. of the bodypart impedances, a more accurate result of the body impedance can be achieved.

For example, the signal processing circuit is configured to determine the corrected body impedance as a difference between the measured body impedance and the error impedance.

In some implementations the signal processing circuit is further configured to determine a first corrected bodypart impedance as a parallel equivalent of the measured first bodypart impedance and the stored first parasitic bodypart impedance, to determine a second corrected bodypart impedance as a parallel equivalent of the measured second bodypart impedance and the stored second parasitic bodypart impedance, and to determine the error impedance based on the first corrected bodypart impedance, the second corrected bodypart impedance and the stored input impedance.

In some implementations the control circuit is further configured to control measurement of a total impedance, where the stimulus current is applied through the third and the fourth terminal, and the input voltage is measured between the third and the fourth terminal. In such implementation the signal processing circuit may be further configured to determine a corrected total impedance as a parallel equivalent of the measured total impedance and a stored parasitic total impedance, and to determine the corrected body impedance further based on the corrected total impedance.

In some implementations the signal processing circuit is further configured to determine the stored first parasitic bodypart impedance, the stored second parasitic bodypart impedance and the stored input impedance during a calibration phase.

For example, during the calibration phase the first, second third and fourth terminals are connected to a calibration body via respective electrodes. The signal processing circuit is configured to determine the stored first parasitic bodypart impedance, the stored second parasitic bodypart impedance and the stored input impedance based on respective measurements and known impedance values of the calibration body corresponding to the measurements. Such calibration may be performed during or at the end of a production phase, such that the corresponding parasitic impedances and the input impedance can be stored in a memory of the bioimpedance measurement circuit.

The electrode arrangement including the first electrode, the second electrode, the third electrode and the fourth electrode respectively connected to the corresponding terminal of the set of terminals may be part of the bioimpedance measurement circuit.

In an embodiment of a bioimpedance measurement method according to the improved bioimpedance measurement concept the bioimpedance measurement method is performed with a set of terminals comprising a first terminal connected to a first electrode being attached to a body, a second terminal connected to a second electrode being attached to the body, a third terminal connected to a third electrode being attached to the body, and a fourth terminal connected to a fourth electrode being attached to the body. The method comprises:
generating a stimulus current with a measurement frequency;
determining a body impedance in response to the stimulus current and a measured input voltage in response to the stimulus current, where the stimulus current is applied through the first and the second terminal, and the input voltage is measured between the third and the fourth terminal;
determining a first bodypart impedance in response to the stimulus current and the measured input voltage, where the stimulus current is applied through the first and the third terminal, and the input voltage is measured between the first and the third terminal;
determining a second bodypart impedance in response to the stimulus current and the measured input voltage, where the stimulus current is applied through the second and the fourth terminal, and the input voltage is measured between the second and the fourth terminal;
determining an error impedance based on the measured first bodypart impedance, the measured second bodypart impedance, a stored first parasitic bodypart impedance, a stored second parasitic bodypart impedance, and a stored input impedance; and
determining a corrected body impedance based on the measured body impedance and the error impedance.

The bioimpedance measurement method may be a computer implemented method that e.g. is carried out by a processor or programmable circuit.

Further implementations of the method become readily apparent for the skilled reader from the various implementations described above in conjunction with the bioimpedance measurement circuit.

According to one embodiment of the improved bioimpedance measurement concept, a computer program product is disclosed, the computer program product comprising instructions which, when executed on one or more processors in connection with a first, a second, a third and a fourth terminal, e.g. as described above, cause the one or more processors to perform the bioimpedance measurement method according to one of the disclosed implementations.

The various implementations of the bioimpedance measurement circuit and method may be used in various applications and products, e.g. electronic devices, like vital sign monitoring in wearables (smartwatches) or healthcare applications like diagnostics. The body impedance can for example be the basis for determination of Body-Cell-Mass Composition (BCM), hydration level detection, calories consumption, fat, muscle percentage, stress level, to name only a few.

### BRIEF DESCRIPTION OF DRAWINGS

The improved bioimpedance measurement concept will be explained in more detail in the following with the aid of the drawings. Elements and functional blocks having the same or similar function bear the same reference numerals throughout the drawings. Hence their description is not necessarily repeated in the following drawings.

In the drawings:

FIG. 1 shows an example implementation of a bioimpedance measurement circuit with asymmetries in the current/voltage paths because of mismatch between the skin/electrode contact impedances;

FIG. 2 shows a further example implementation of a bioimpedance measurement circuit;

FIG. 3 shows a detail of the bioimpedance measurement circuit of FIG. 2;

FIGS. 4a) to 4f) show various measurement configurations for a calibration phase of a bioimpedance measurement circuit;

FIGS. 5a) to 5d) show various measurement configurations for a measurement phase of a bioimpedance measurement circuit;

FIG. 6 shows a detail of the bioimpedance measurement circuit used in FIG. 5 and FIG. 6;

FIG. 7 shows an example block diagram of operational phases in a bioimpedance measurement circuit;

FIG. 8 shows another example block diagram of operational phases in a bioimpedance measurement circuit; and

FIG. 9 shows another example implementation of a bioimpedance measurement circuit.

### DETAILED DESCRIPTION

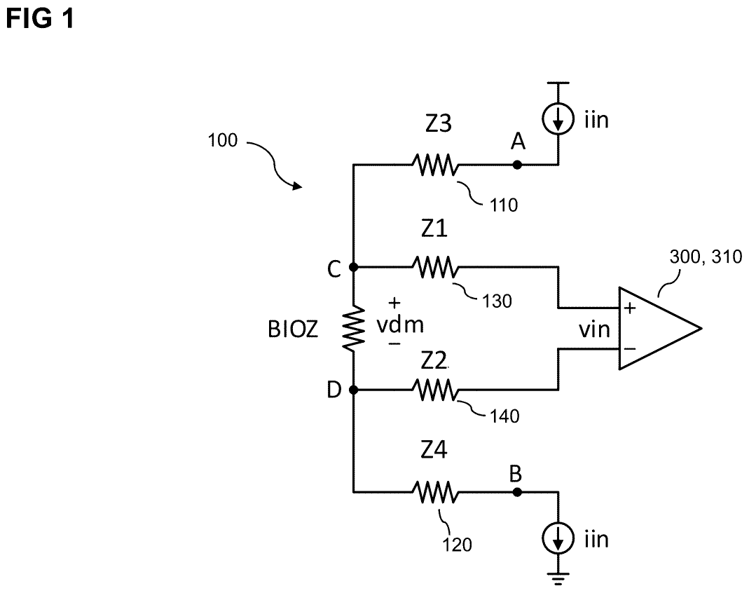

FIG. 1 illustrates a bioimpedance measurement circuit that is based on a four point measurement method. The bioimpedance measurement circuit comprises an electrode arrangement 100 for attaching to a human body. The electrode arrangement 100 includes a first pair of electrodes 110, 120 to apply a stimulus current iin. The electrodes 110, 120 are attached to nodes A and B of a human body. It is assumed that electrode 110 has an electrode contact impedance Z3, and electrode 120 has an electrode contact impedance Z4. The electrode arrangement further comprises a second pair of electrodes 130, 140 to measure a voltage vin between the electrodes 130, 140 of the second pair. The electrodes 130, 140 are attached to nodes C and D of a human body. It is assumed that electrode 130 has an electrode contact impedance Z1, and electrode 140 has an electrode contact impedance Z2.

The electrodes 110, 120, 130 and 140 are placed to measure a bioimpedance BIOZ of interest between nodes C and D of the human body or between electrodes 130 and 140. The generated voltage vin is applied to and evaluated by an evaluation circuit 300 which is illustrated in simplified form as a differential amplifier 310 in FIG. 1. The amplitude and phase of the resulting voltage signal vin depends on the bioimpedance BIOZ of interest, and the bioimpedance BIOZ is the quotient vin/iin.

Ideally, the voltage vin at the input of the evaluation circuit 300 is equal to the desired differential signal vdm which represents the voltage drop across the bioimpedance BIOZ. In reality, however, due to asymmetries in the current path a common mode signal is created across the body impedance BIOZ. Assuming that in some implementations, the common mode at nodes A and B is regulated to be some internal defined reference voltage vref, the common mode signal across nodes C and D is:

$$\frac{VC + VD}{2} = \frac{VA + VB}{2} + \frac{Z 4 - Z 3}{2} \cdot iin = vref + k \cdot iin$$

where VA is the voltage potential at node A, VB is the voltage potential at node B, VC is the voltage potential at node C, and VD is the voltage potential at node D.

As a result, a common mode signal vcm is generated across the bioimpedance BIOZ between nodes C and D, wherein the common mode signal vcm is proportional to the stimulus current:

$$vcm = \frac{Z 4 - Z 3}{2} \cdot iin$$

Due to asymmetries in the voltage path, the common mode signal vcm is translated to an unwanted extra differential signal vdmx. Those asymmetries arise because of mismatch between the skin/electrode contact impedances.

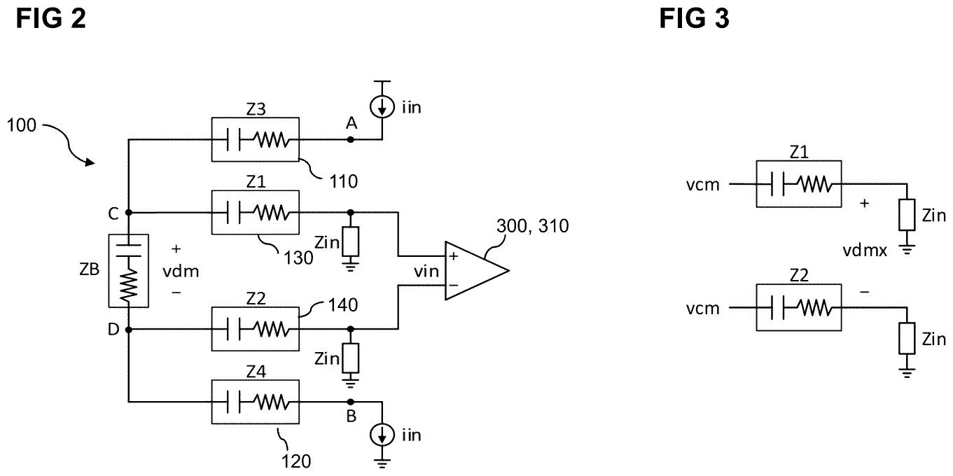

FIG. 2 shows a further example implementation of a bioimpedance measurement circuit, which is similar to the implementation of FIG. 1 and includes input impedances Zin at the inputs of amplifier 310. Furthermore, it can be seen that the impedances at the electrodes 110, 120, 130, 140 have both a resistive and a capacitive component.

FIG. 3 shows a detail of the bioimpedance measurement circuit of FIG. 2. Let Zin be the single ended input impedance of the amplifier 310. It is expected that this impedance is mainly capacitive, but in general it can be modeled as a parallel combination of a resistor and a capacitor:

$$Zin = \frac{Rin}{1 + j \cdot ω \cdot Rin \cdot Cin}$$

Due to the mismatch of the contact impedances, the common mode signal in the body will be translated to a differential signal vdmx at the input of the amplifier 310:

$$vdmx = \frac{Zin}{Z 1 + Zin} \cdot vcm - \frac{Zin}{Z 2 + Zin} \cdot vcm$$

Using the expression for vcm obtained above vdmx results to:

$$vdmx = ( \frac{Zin \cdot ( Z 2 - Z 1 )}{( Z 1 + Zin ) \cdot ( Z 2 + Zin )} ) \cdot ( \frac{Z 4 - Z 3}{2} ) \cdot iin$$

Referring back to FIG. 2, let Zb be the body impedance, then assuming for now no significant attenuation in the differential path, the expected measured differential voltage vdm is:

$$vdm = iin \cdot Zb$$

However, due to the error of the common mode signal the total input differential voltage will be:

$$vin = vdm + vdmx , or$$

$$vin = iin \cdot Zb + ( \frac{Zin \cdot ( Z 2 - Z 1 )}{( Z 1 + Zin ) \cdot ( Z 2 + Zin )} ) \cdot ( \frac{Z 4 - Z 3}{2} ) \cdot iin$$

Then the measured (body) impedance will be:

$$Zmeas = \frac{vin}{iin} = Zb + ( \frac{Zin \cdot ( Z 2 - Z 1 )}{( Z 1 + Zin ) \cdot ( Z 2 + Zin )} ) \cdot ( \frac{Z 4 - Z 3}{2} ) = Zb + Zerr$$

The final expression for the error impedance Zerr is then:

$$Zerr = ( \frac{Zin \cdot ( Z 2 - Z 1 )}{( Z 1 + Zin ) \cdot ( Z 2 + Zin )} ) \cdot ( \frac{Z 4 - Z 3}{2} )$$

Assuming that the contact impedances at both inputs of the amplifier 310 are equal, i.e. for a first bodypart impedance, e.g. a wrist impedance Zw, and a second bodypart impedance, e.g. a finger impedance Zf,

$$Z 3 \approx Z 1 = Zw and$$

$$Z 4 \approx Z 2 = Zf ,$$

the error impedance expression for Zerr becomes:

$$Zerr = \frac{1}{2} \cdot ( \frac{Zin \cdot {( Zf - Zw )}^{2}}{( Zw + Zin ) \cdot ( Zf + Zin )} )$$

Hence the corrected body impedance results as a difference between the measured body impedance Zmeas and the error impedance Zerr.

For example, the correction method according to the improved bioimpedance measurement concept includes estimating by measurement and calibration, the values of Zf, Zw and Zin, calculate the error impedance Zerr and subtract it from the measured body impedance.

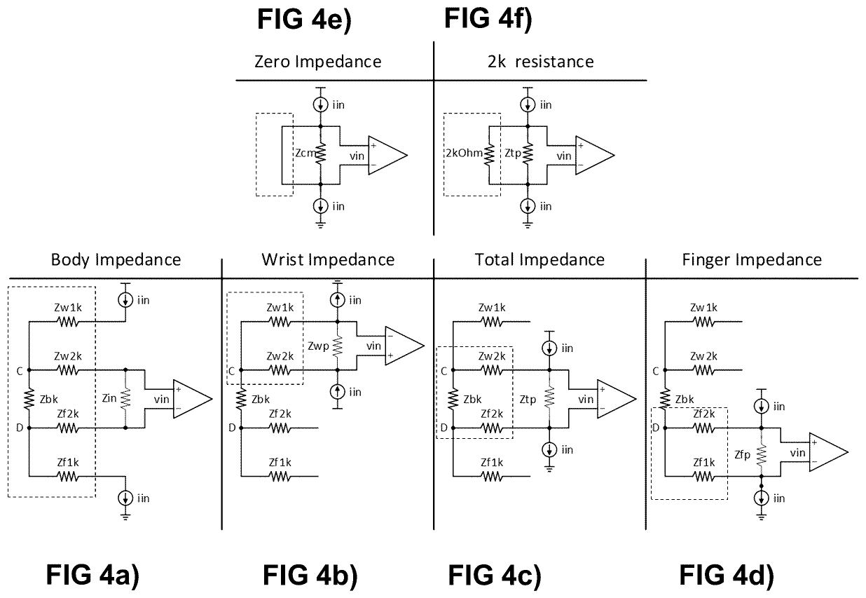

FIGS. 4a) to 4f) show various measurement configurations for a calibration phase of a bioimpedance measurement circuit. As described, there is a dependence of the error impedance Zerr on the first bodypart impedance or wrist impedance Zw, on the second bodypart impedance or finger impedance Zf and on the input impedance Zin, the values of which have to be known for each measurement of the body impedance. While the input impedance Zin of the amplifier can be assumed to remain the same, the effective bodypart impedances may change with e.g. changing application of the electrodes.

For example, in the configuration of FIG. 4a) for measuring a body impedance, the stimulus current iin is applied through the first and the second terminal, and the input voltage vin is measured between the third and the fourth terminal or the corresponding electrodes, respectively. Similarly, in the configuration of FIG. 4b) for measuring a first bodypart impedance or wrist impedance Zw, the stimulus current iin is applied through the first and the third terminal, and the input voltage vin is measured between the first and the third terminal. In the configuration of FIG. 4c) a total impedance Zt can be measured, where the stimulus current iin is applied through the third and the fourth terminal, and the input voltage vin is measured between the third and the fourth terminal. Similarly, in the configuration of FIG. 4d) for measuring a second bodypart impedance or finger impedance Zf, the stimulus current iin is applied through the second and the fourth terminal, and the input voltage vin is measured between the second and the fourth terminal or the corresponding electrodes, respectively.

During the calibration phase, the electrodes are placed on a body or other object with known impedances like a known body impedance Zbk, known wrist impedances Zw1k, Zw2k and known finger impedances Zf1k, Zf2k.

In the configurations of FIG. 4e) and FIG. 4f) a zero impedance and a known resistance impedance like 2kΩ are measured.

For example, ideally, with the configuration of FIG. 4b) it is possible to measure directly 2*Zw. However when the magnitude of 2*Zw is high enough, the current leakage through a parasitic parallel path can cause significant error. In order to correct this error it is proposed to have a calibration phase, where the impedance Zwp of the parallel parasitic path is estimated.

During the calibration phase a known reference impedance is used, so we can know in advance the value of the impedance.

Let Zw1k, Zw2k be the known wrist impedances of the selected reference impedance for calibration. So, due to the parasitic parallel path, instead of measuring Zw1k+Zw2k the system will measure a different value that we will denote as Zwm.

The measured contact impedance will be

$$Zwm = ( Zw 1 k + Zw 2 k ) ‖ Zwp$$

By using known impedances Zw1k, Zw2k, we can calculate the parasitic impedance by solving the equation to:

$$Zwp = \frac{Zwm ( Zw 1 k + Zw 2 k )}{Zw 1 k + Zw 2 k - Zwm}$$

Similar approaches for the other configurations will readily be apparent to the skilled reader

In general, during calibration the known reference impedances are connected, and four values are measured:
Body impedance Zb
Wrist impedance Zw (divide the result by 2 to get Zw)
Finger impedance Zf (divide the result by 2 to get Zf)
Total impedance Zt (optional)

The known values of the reference impedances will be denoted as:
Known body impedance: Zbk
Known wrist impedance: Zw1k, Zwk2
Known finger impedance: Zf1k, Zf2k
Known total impedance: Ztk=Zbk+Zw2k+Zf2k

Based on the previous information, 4 values are calculated
Equivalent impedance in parallel with wrist: Zwp
Equivalent impedance in parallel with finger: Zfp
Equivalent impedance in parallel with total: Ztp (optional)
Equivalent single ended input impedance: Zin

Equations e.g. used during calibration:

Intermediate parameter fac (Current division factor for total impedance):

$$fac = \frac{Ztp}{Ztp + Ztk}$$

Intermediate parameter Zberr (Body impedance error):

$$Zberr = \frac{Zb}{fac} - Zbk$$

For determining the input impedance Zin the quadratic equation of the error impedance Zerr is resolved:

$$a = 2 \cdot Zberr$$

$$b = 2 \cdot Zberr \cdot ( Zfk + Zwk ) - {( Zfk - Zwk )}^{2}$$

$$c = 2 \cdot Zberr \cdot Zfk \cdot Zwk$$

$$delta = {b}^{2} - 4 \cdot a \cdot c$$

$$Zin = \frac{- b + \sqrt{delta}}{2 \cdot a}$$

If imaginary part of Zin is positive then

$$Zin = \frac{- b - \sqrt{delta}}{2 \cdot a}$$

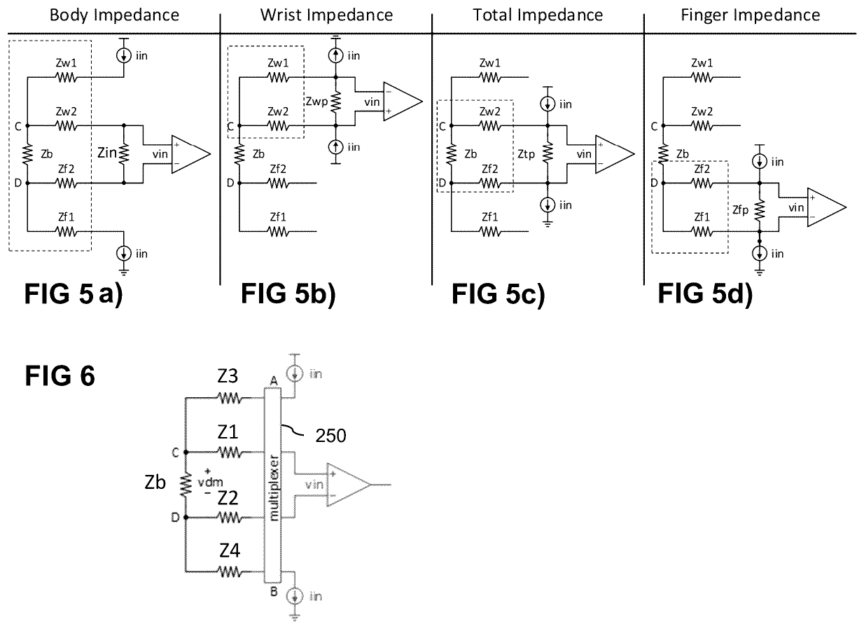

Referring now to FIGS. 5a) to 5d), various measurement configurations for a measurement phase of a bioimpedance measurement circuit are shown. The respective configurations of FIGS. 5a) to 5d), which for example can be achieved employing a multiplexer 250 as shown in FIG. 6, correspond to those described for FIGS. 4a) to 4d). The only difference is that the respective impedances are not known. However, at this stage the parasitic impedances are available for performing the corrections.

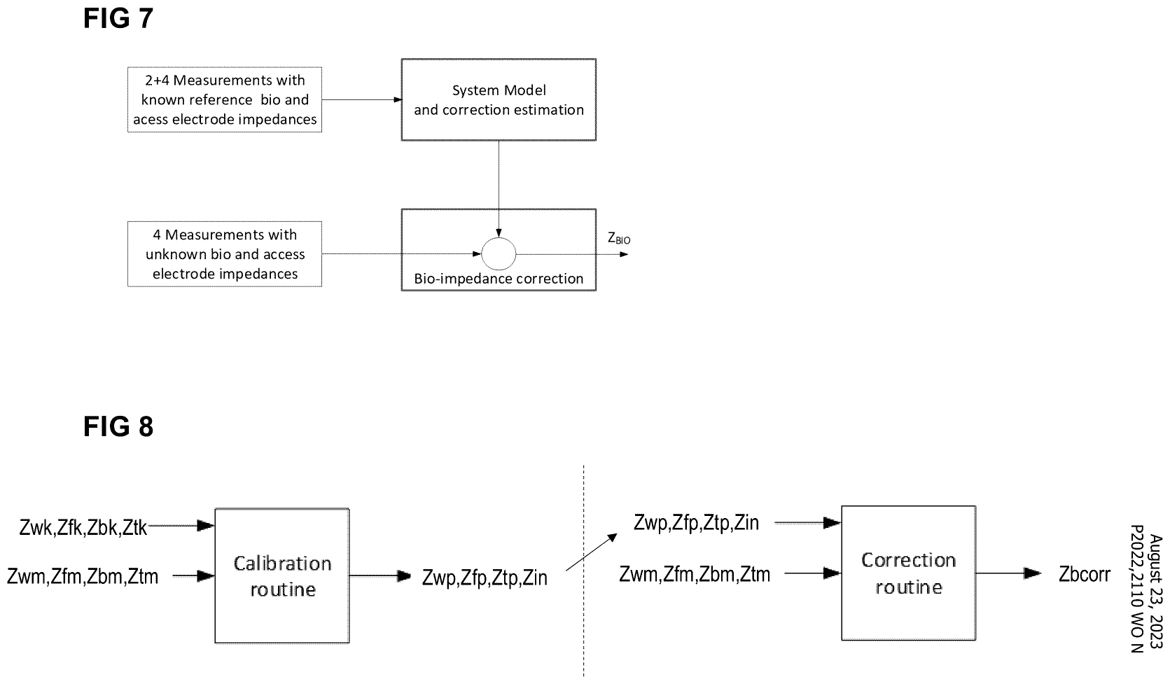

FIG. 7 shows an example block diagram of operational phases in a bioimpedance measurement circuit. For example, the system model and correction estimation corresponds to the calibration phase having as its input the 2+4 measurements and the known impedances, as described above. During the regular operation or measurement operation, only the four measurements of FIGS. 5a) to 5d) are made, which form the basis for the actual determination of the body impedance, as also described above.

As shown in FIG. 8, the calibration routine receives the measured values Zw, Zf, Zt, Zb, wherein Zt may be optional, for the known body, together with the know impedances Zwk, Zfk, Zbk, Ztk. As described, the result is the parasitic impedances Zwp, Zfp, Ztp and the input impedance Zin. During the regular operation or measurement operation the correction routine (also) receives the measured values Zw, Zf, Zt, Zb, wherein Zt may be optional, and performs the respective corrections based on the parasitic impedances Zwp, Zfp, Ztp and the input impedance Zin in order to determine the corrected body impedance Zb.

Equations used during measurement and correction:

Let's denote the measured values as Zwm, Zfm, Zbm, Ztm. Let's denote corrected values as Zwc, Zfc, Zbc, Ztc. Corrected impedances:

$$Zwc = Zwm ‖ ( - Zwp )$$

$$Zfc = Zfm ‖ ( - Zfp )$$

$$Ztc = Ztm ‖ ( - Ztp )$$

The corrected impedances Zwc and Zfc, together with Zin, need to be used for calculating the error impedance Zerr as disclosed above, for each measurement.

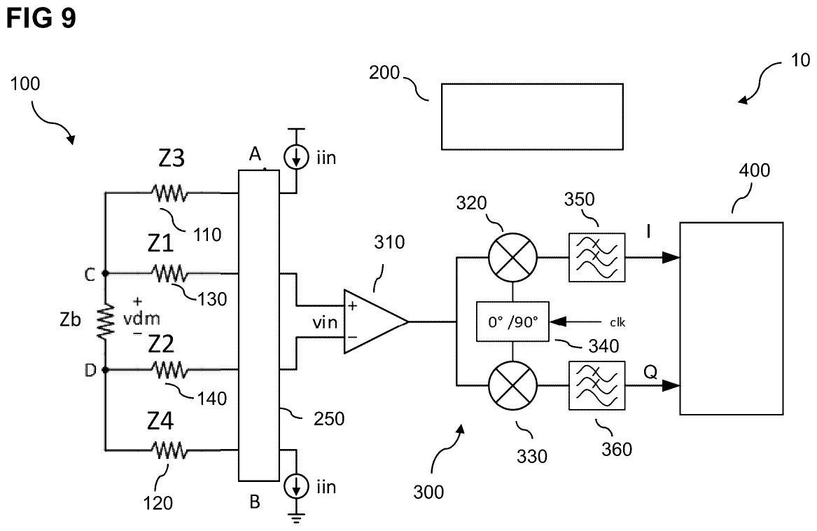

A bioimpedance measurement circuit 10 which allows to determine the real part I and the imaginary part Q of a measured bioimpedance is shown in FIG. 9. The measurement circuit 10 comprises an electrode arrangement 100 for attaching to a body. The electrode arrangement 100 includes four electrodes 110, 120, 130, 140 to apply a stimulus current iin, and to measure an input voltage vin, depending on a configuration selected via multiplexer 250. The electrodes 110, 120 are attached to nodes A and B of a human body, wherein Z3 denotes an electrode contact impedance between the skin of the body and the electrode 110, and Z4 denotes an electrode contact impedance between the skin of the body and the electrode 120.

The bioimpedance measurement circuit 10 further comprises a control circuit 200 to control respective impedance measurements, and an evaluation circuit 300. The evaluation circuit 300 is configured for determining a real part I and an imaginary part Q of the measured impedance in response to the stimulus current iin and the measured input voltage vin.

The evaluation circuit 300 comprises a differential amplifier 310 to apply the measured input voltage vin. An output side of the differential amplifier 310 is coupled to a first path comprising a modulator 320 and a low pass filter 350, and a second path comprising a modulator 330 and a low pass filter 360. The modulators 320 and 330 are coupled to an oscillator 340. The arrangement of modulators 320, 330 coupled to oscillator 340 with low pass filters 350, 360 arranged behind the modulators 320, 330 is provided to implement a quadrature demodulation which allows to measure the real part I and the imaginary part Q of the measured impedance.

Referring to FIG. 9, the bioimpedance measurement circuit comprises a signal processing circuit 400, e.g. a processor for determining a corrected value of the bioimpedance (BIOZ), i.e. for performing the corresponding calculations as described above. To this end the signal processing circuit 400 may comprise or be connected to a memory for storing the values needed for correction.

The control circuit 200 is configured to control the measurement of the impedances by applying the stimulus current iin with a measurement frequency F to the electrode arrangement 100, in particular by controlling the multiplexer 250 according to the desired impedance.

The control circuit 200 may also control the different operating modes, i.e. calibration operation and regular operation or measurement operation, respectively. The control circuit 200 itself may be controlled by the signal processing circuit 400.

The embodiments of the improved bioimpedance measurement concept disclosed herein have been discussed for the purpose of familiarizing the reader with novel aspects of the implementation of the improved bioimpedance measurement concept. Although preferred embodiments have been shown and described, many changes, modifications, equivalents and substitutions of the disclosed concepts may be made by one having skill in the art without departing from the scope of the claims.

In particular, the implementation of the improved bioimpedance measurement concept is not limited to the disclosed embodiments, and gives examples of many alternatives possible for the features included in the embodiments discussed. However, it is intended that any modifications, equivalents and substitutions of the disclosed concepts be included within the scope of the claims which are appended hereto.

Features recited in separate dependent claims may be advantageously combined. Moreover, reference signs used in the claims are not limited to be construed as limiting the scope of the claims.

Furthermore, as used herein, the term “comprising” does not exclude other elements. In addition, as used herein, the article “a” is intended to include one or more than one component or element, and is not limited to be construed as meaning only one.

1. A bioimpedance measurement circuit, comprising:
a set of terminals comprising:
a first terminal for connecting a first electrode to be attached to a body;
a second terminal for connecting a second electrode to be attached to the body;
a third terminal for connecting a third electrode to be attached to the body; and
a fourth terminal for connecting a fourth electrode to be attached to the body;
a control circuit to control application of a stimulus current with a measurement frequency through a first subset of two selected terminals of the set of terminals and measurement of an input voltage in response to the stimulus current at a second subset of two selected terminals of the set of terminals;
an evaluation circuit for determining a measured impedance in response to the stimulus current and the measured input voltage; and
a signal processing circuit for determining a body impedance;
wherein the control circuit is configured to control measurement of:
the body impedance, where the stimulus current is applied through the first and the second terminal, and the input voltage is measured between the third and the fourth terminal;
a first bodypart impedance, where the stimulus current is applied through the first and the third terminal, and the input voltage is measured between the first and the third terminal; and
a second bodypart impedance, where the stimulus current is applied through the second and the fourth terminal, and the input voltage is measured between the second and the fourth terminal; and
wherein the signal processing circuit is configured to:
determine an error impedance based on the measured first bodypart impedance, the measured second bodypart impedance, a stored first parasitic bodypart impedance, a stored second parasitic bodypart impedance, and a stored input impedance; and
determine a corrected body impedance based on the measured body impedance and the error impedance.

2. The bioimpedance measurement circuit according to claim 1, wherein the signal processing circuit is configured to determine the corrected body impedance as a difference between the measured body impedance and the error impedance.

3. The bioimpedance measurement circuit according to claim 1, wherein the signal processing circuit is further configured to:
determine a first corrected bodypart impedance as a parallel equivalent of the measured first bodypart impedance and the stored first parasitic bodypart impedance;
determine a second corrected bodypart impedance as a parallel equivalent of the measured second bodypart impedance and the stored second parasitic bodypart impedance; and
determine the error impedance based on the first corrected bodypart impedance, the second corrected bodypart impedance and the stored input impedance.

4. The bioimpedance measurement circuit according to claim 1, wherein the control circuit is further configured to control measurement of a total impedance, where the stimulus current is applied through the third and the fourth terminal, and the input voltage is measured between the third and the fourth terminal, and wherein the signal processing circuit is further configured to determine a corrected total impedance as a parallel equivalent of the measured total impedance and a stored parasitic total impedance, and to determine the corrected body impedance further based on the corrected total impedance.

5. The bioimpedance measurement circuit according to claim 1, wherein the signal processing circuit is further configured to determine the stored first parasitic bodypart impedance, the stored second parasitic bodypart impedance and the stored input impedance during a calibration phase.

6. The bioimpedance measurement circuit according to claim 5, wherein during the calibration phase the first, second third and fourth terminals are connected to a calibration body via respective electrodes, and wherein the signal processing circuit is configured to determine the stored first parasitic bodypart impedance, the stored second parasitic bodypart impedance and the stored input impedance based on respective measurements and known impedance values of the calibration body corresponding to the measurements.

7. The bioimpedance measurement circuit according to claim 1, further comprising an electrode arrangement for attaching to a body, the electrode arrangement including the first electrode, the second electrode, the third electrode and the fourth electrode respectively connected to a corresponding terminal of the set of terminals.

8. An electronic device comprising the bioimpedance measurement circuit according to claim 1.

9. A bioimpedance measurement method being performed with a set of terminals, comprising:
a first terminal connected to a first electrode being attached to a body;
a second terminal connected to a second electrode being attached to the body;
a third terminal connected to a third electrode being attached to the body; and
a fourth terminal connected to a fourth electrode being attached to the body;
the method comprising:
generating a stimulus current with a measurement frequency;
determining a body impedance in response to the stimulus current and a measured input voltage in response to the stimulus current, where the stimulus current is applied through the first and the second terminal, and the input voltage is measured between the third and the fourth terminal;
determining a first bodypart impedance in response to the stimulus current and the measured input voltage, where the stimulus current is applied through the first and the third terminal, and the input voltage is measured between the first and the third terminal;
determining a second bodypart impedance in response to the stimulus current and the measured input voltage, where the stimulus current is applied through the second and the fourth terminal, and the input voltage is measured between the second and the fourth terminal;
determining an error impedance based on the measured first bodypart impedance, the measured second bodypart impedance, a stored first parasitic bodypart impedance, a stored second parasitic bodypart impedance, and a stored input impedance; and
determining a corrected body impedance based on the measured body impedance and the error impedance.

10. The method according to claim 9, wherein the corrected body impedance is determined as a difference between the measured body impedance and the error impedance.

11. The method according to claim 9, wherein determining the error impedance comprises:
determining a first corrected bodypart impedance as a parallel equivalent of the measured first bodypart impedance and the stored first parasitic bodypart impedance;
determining a second corrected bodypart impedance as a parallel equivalent of the measured second bodypart impedance and the stored second parasitic bodypart impedance; and
determining the error impedance based on the first corrected bodypart impedance, the second corrected bodypart impedance and the stored input impedance.

12. The method according to claim 9, further comprising:
determining a total impedance in response to the stimulus current and the measured input voltage, where the stimulus current is applied through the third and the fourth terminal, and the input voltage is measured between the third and the fourth terminal;
determining a corrected total impedance as a parallel equivalent of the measured total impedance and a stored parasitic total impedance; and
determining the corrected body impedance further based on the corrected total impedance.

13. The method according to claim 9, wherein determining the stored first parasitic bodypart impedance, the stored second parasitic bodypart impedance and the stored input impedance is performed during a calibration phase.

14. The method according to claim 13, wherein during the calibration phase the first, second third and fourth terminals are connected to a calibration body via respective electrodes, and wherein the stored first parasitic bodypart impedance, the stored second parasitic bodypart impedance and the stored input impedance are determined based on respective measurements and known impedance values of the calibration body corresponding to the measurements.

15. A computer program product comprising instructions which, when executed on one or more processors in connection with the first terminal, the second terminal, the third terminal, and the fourth terminal, cause the one or more processors to perform the bioimpedance measurement method according to claim 9.

16. The bioimpedance measurement circuit according to claim 1, wherein the signal processing circuit is configured to determine the error impedance according to:
$$Zerr = \frac{1}{2} \cdot ( \frac{Zin \cdot {( Zf - Zw )}^{2}}{( Zw + Zin ) \cdot ( Zf + Zin )} ) ,$$
wherein Zerr is the error impedance, Zin is the stored input impedance, Zf is the measured second bodypart impedance, and Zw is the measured first bodypart impedance.

17. The method according to claim 9, wherein the error impedance is determined according to:
$$Zerr = \frac{1}{2} \cdot ( \frac{Zin \cdot {( Zf - Zw )}^{2}}{( Zw + Zin ) \cdot ( Zf + Zin )} ) ,$$
wherein Zerr is the error impedance, Zin is the stored input impedance, Zf is the measured second bodypart impedance, and Zw is the measured first bodypart impedance.
